(19) United States
(12) Patent Application Publication
(10) Pub. No.: US 2021/0033549 A1
(43) Pub. Date: Feb. 4, 2021

(54) FULL-VIEW-FIELD QUANTITATIVE STATISTICAL DISTRIBUTION REPRESENTATION METHOD FOR MICROSTRUCTURES of y' PHASES IN METAL MATERIAL

(71) Applicant: CENTRAL IRON AND STEEL RESEARCH INSTITUTE, Beijing (CN)

(72) Inventors: Weihao Wan, Beijing (CN); Dongling Li, Beijing (CN); Haizhou Wang, Beijing (CN); Lei Zhao, Beijing (CN); Xuejing Shen, Beijing (CN); Yunhai Jia, Beijing (CN); Bing Han, Beijing (CN); Jie Li, Beijing (CN); Yuhua Lu, Beijing (CN)

(21) Appl. No.: 17/067,794

(22) Filed: Oct. 12, 2020

(30) Foreign Application Priority Data
Jun. 22, 2020 (CN) 202010575011.8

(51) Int. Cl. G01N 23/2251; G06N 20/00; G06T 7/10

(52) U.S. Cl. CPC G01N 23/2251; G06N 20/00; G06T 7/10; G01N 2223/605; G06T 2207/20081; G01N 2223/418; G01N 2223/405; G01N 2223/305; G01N 2223/401

(57) ABSTRACT

The present invention discloses, a full-view-field quantitative statistical distribution representation method for microstructures of γ′ phases in a metal material, comprising the following steps: step a: labeling γ′ phases, cloud clutters and γ matrixes by Labelme, and then making standard feature training samples; step b: building a deep learning-based feature recognition and extraction model by means of BDU-Net; step e: collecting γ′ feature maps in the metal material to be detected; step d: automatically recognizing and extracting the γ′ phases; and step e: performing in-situ quantitative statistical distribution representation on the γ phases in the full view field within a large range. The full-view-field quantitative statistical distribution representation method for microstructures of γ′ phases in a metal material provided by the present invention realizes automatic, high-speed and high-quality recognition and extraction of features of γ phases in the metal material

### TECHNICAL FIELD

The present invention relates to the technical field of detection and recognition of γ′ phases in metal materials, in particular to a full-view-field, quantitative statistical distribution representation method for microstructures of γ′ phases in a metal material.

### BACKGROUND

Phases in a metal material that are distributed in a matrix in a discontinuous state and cannot be surrounded, by other phases are collectively called precipitated phases. There is a clear interface between the precipitated phases and the matrix structure, so the precipitated phases play a very important role in steel, and have important influence on the strength, toughness, plasticity, deep drawability, fatigue, attrition, fracture, corrosion and many important physical and chemical properties of steel. For examples, two basic constituent phases, of the precipitation hardening nickel-based superalloy are γ phase and γ′ phase, the γ′ phase is the most important precipitated phase thereof, wherein the γ′ phases in the single crystal nickel-based superalloy exist in a square-like form, and the area fraction, distribution, size and morphology of γ′ phase particles are key factors affecting alloy mechanical properties, especially high-temperature properties. Therefore, the statistical quantitative distribution analysis of γ′ phases in the metal material is of great significance for the study of the metal material.

At present, the feature maps of γ′ phases are mainly acquired by an SEM at high magnification, and the statistics of the morphology, area fraction, distribution and size and other information of γ′ phases are mainly performed by image processing software such as Image-pro Plus, Photoshop, etc., and the feature maps are parsed by relevant algorithms to obtain the sizes of the particles and calculate area fractions. However, the above methods are all used to process a few features, and manual methods are used for post-processing so that the processing results can meet the requirements of quantitative statistics. This statistical method can only be used for performing statistical analysis on several hundred to several thousand γ′-phase features, while for a single crystal superalloy sample greater than φ10 mm, the number of γ′ phases therein has exceeded 1 billion, and the statistical information accounts for a small proportion in the global information and is not representative enough. For such method, not only the statistical efficiency is low, but also because the non-homogeneity essence of material decides that such measurement mode lacks of statistical representativeness, it is difficult to guarantee the accuracy, and is unable to meet the requirements of quantitative statistical distribution representation of γ′ phases in the single crystal superalloy within a large range.

The traditional SEM technology can't support full-view-field and high-throughput calculation in the aspect of feature map data algorithms for processing microstructures. The method frequently used to realize segmentation of microstructures in SEM maps by image processing software such as Image-Pro Plus, etc. can only be used to process a limited number of features in a few view fields. Usually, only hundreds to thousands of microstructure features can be statistically analyzed, and only partial statistical information can be obtained. For a macroscopic metal material, it is a collection of uneven microstructures in essence, and the observation of microstructures in a single view field or partial multiple view fields cannot reflect the distribution features of the overall microstructure of the material. In order to find the correlation between microstructures at different scales, accurate positioning adds extra workload.

### SUMMARY

The object of the present invention is to provide a full-view-field quantitative statistical distribution representation method for microstructures of γ′ phases in a metal material to realize automatic, high-speed and high-quality recognition and extraction of features of γ′ phases in the metal material and full-view-field in-situ quantitative statistical distribution representation of the features based on the depth learning theory, and overcome the defects of small view field, few features and insufficient representativeness of the traditional statistical method for γ′ phases.

In order to achieve the above object, the present invention provides the following solution:

A full-view-field quantitative statistical distribution representation method for microstructures of γ′ phases in a metal material, comprising the following steps:

a) performing metallographic sample preparation, polishing and chemical etching on standard metal material samples with the same material as a metal material to be detected, randomly sampling and shooting the processed standard metal material samples by a scanning electron microscope at high magnification, and building a -γ′-phase feature map data set; labeling γ′ phases, cloud clutters and γ matrixes by Labelme, and then making standard feature training samples;

b) optimizing a deep learning-based image segmentation network U-Net, building a feature recognition and extraction network BDU-Net, performing data augmentation on the standard feature training samples, dividing the augmented data into a training set and a validation set, training with the training set, taking the MPA of the validation set as a judgment condition of training termination, saving parameters after the training is terminated, and saving the trained network as a final feature recognition and extraction model;

c) performing metallographic sample preparation, polishing and chemical etching on the metal material to be detected, and performing automatic collection of large-sized full-view-field γ′-phase feature maps on the surface of the processed metal material to be detected by a Navigator-OPA high-throughput scanning electron microscope;

d) inputting the y′-phase feature maps obtained in the step c into the feature recognition and extraction model built in the step b, and thus obtaining binary

Description images with γ′ phases labeled in situ; and

e) processing the binary images obtained in the step d by the connected component algorithm, acquiring the size, area and position information of each γ′ phase, mining the statistical results, selecting appropriate regions as calculation units, calculating the area fractions of γ′ phases of different sizes on each calculation unit, and studying the distribution of the γ′ phases of different sizes in the full view field.

Optionally, in the step c, the number of the automatically collected γ′-phase feature maps is more than 10000.

Optionally, in the step b, the feature recognition and extraction network is a new feature recognition network BDU-Net proposed by adding a connection between blocks on the basis of the J-Net, the BD-U-Net including nine blocks respectively connected by ten maximum pooling layers and ten transposed convolution layers, each block internally consisting of two convolution layers, two ReLu activation functions and one Dropout layer.

Optionally, in the step b, further comprising: preprocessing images containing γ′ phases in the standard feature map data set, specifically including translation, rollover, zooming-in/out, rotation and increase in noise.

Optionally, in the step d, when the binary images of the γ′-phase feature maps are extracted using a view field with a pixel of 12288*12288 the time duration consumed in the extraction process is 12.5s.

Optionally, in the step e, the size, area and position of 14400 γ′ phases are obtained respectively by means of the connected component algorithm, and are statistically analyzed, to obtain statistical results.

Optionally, in the step e, the statistical results are mined, regions of 2.56 μm*2.56 μm are selected as calculation units, and the area fractions of the γ′ phases of different sizes on each calculation unit are calculated.

Optionally, in the step e, further comprising: visualizing the in-situ distribution of γ′ phases of different sizes in the full view field, and observing that the γ′ phases of small sizes are distributed in the dendrite trunk position and the γ′ phases of large sizes are distributed in the interdendritic position.

According to the specific embodiment provided by the present invention, the present invention discloses the following technical effects: compared with the prior art, the full-view-field quantitative statistical distribution representation method for microstructures of γ′ phases in a metal material provided by the present invention has the following advantageous effects:

Firstly, the current statistical method for γ′ phases is mainly used to measure various parameters of γ′ phases through image processing software such as Image-Pro Plus, PhotoShop, etc., and the recognition and extraction of γ′ phases and the measurement and statistics of size, area and other parameters of γ′ phases are completed in a mode of combination of a frequently-used image processing algorithm with manual correction, resulting in heavy workload and low efficiency. The method of the present invention realizes automatic and rapid recognition and extraction of a large number of γ′ phases in the view field and automatic statistics of various parameters of γ′ phases through the combination of a deep learning-based image segmentation and extraction algorithm with a statistical algorithm, greatly improving the efficiency of recognition and statistics, and the method of the present invention has good generalization ability, having high accuracy guarantee when extracting feature maps obtained at different illumination intensities or different batches;

Secondly, in the present invention, the correlation between different blocks is strengthened based on the existing deep learning-based image segmentation algorithm U-Net, a new feature recognition network BD U-Net is proposed, so the phenomena of loss of feature information caused by too deep neural network and gradient disappearance that may occur in the process of back propagation are avoided, the fusion degree of features of different scales and levels is deepened, and the utilization rate of different features is improved; and

Thirdly, the current statistical method for γ′ phases in single crystal superalloys is mainly used to count the number and rough area fraction of γ′ phases in partial small view fields, or accurately measure the size, area and other parameters of γ′ phases with respect to the features of a few γ′ phases; by means of the deep learning and statistics-based method of the present invention, relevant parameters of all γ′ phase features can be quickly extracted under the condition of ensuring higher accuracy, various parameters can be recorded in corresponding positions in the full view field, so that analysis can be performed globally and detailed local analysis can be performed on any region, and thus the statistical information is more comprehensive and abundant; because there are records of position information and corresponding statistical infoniiation, the feature infoniiation can be traced back to the original features quickly and accurately from analysis results, making the analysis results more reliable and representative.

### DESCRIPTION OF DRAWINGS

To more clearly describe the technical solutions in the embodiments of the present invention or in prior art, the drawings required to be used in the embodiments will be simply presented below Apparently, the drawings in the following description are merely some embodiments of the present invention, and for those skilled in the art, other drawings can also be obtained according to these drawings without contributing creative labor.

FIG. 1a is a diagram of the U-Net;

FIG. 1b is a diagram of the BD U-Net;

FIG. 2a shows a test image;

FIG. 2b shows a segmentation result obtained by the U-Net;

FIG. 2c shows a result of post-processing the result obtained by the U-Net;

FIG. 2d shows a segmentation result obtained directly by the BD U-Net;

FIG. 3a shows an image to be labeled;

FIG. 3b shows an image labeled manually;

FIG. 4 is a flow chart of training, extraction and statistics of the feature recognition network of the present invention;

FIG. 5a shows a real labeled image,

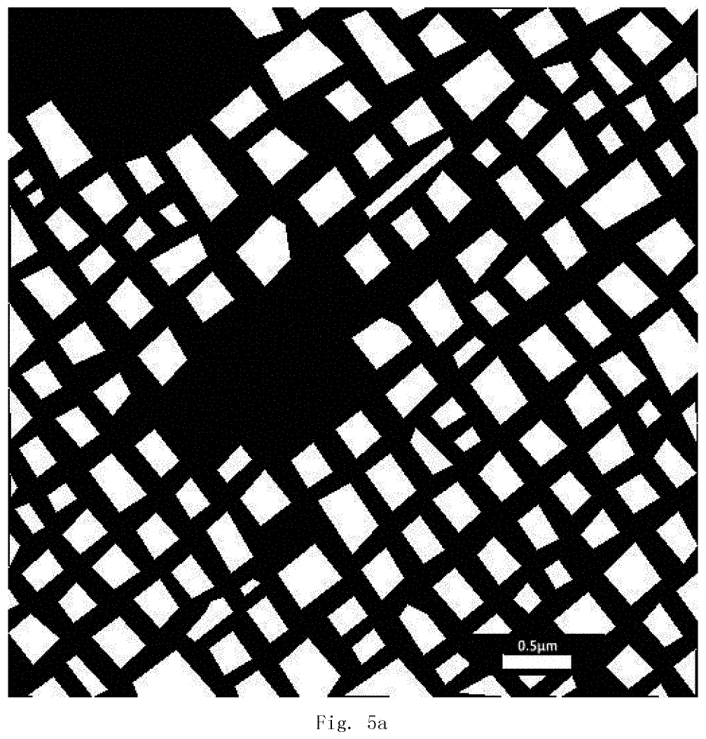

FIG. 5b shows augmented image data obtained by rotation;

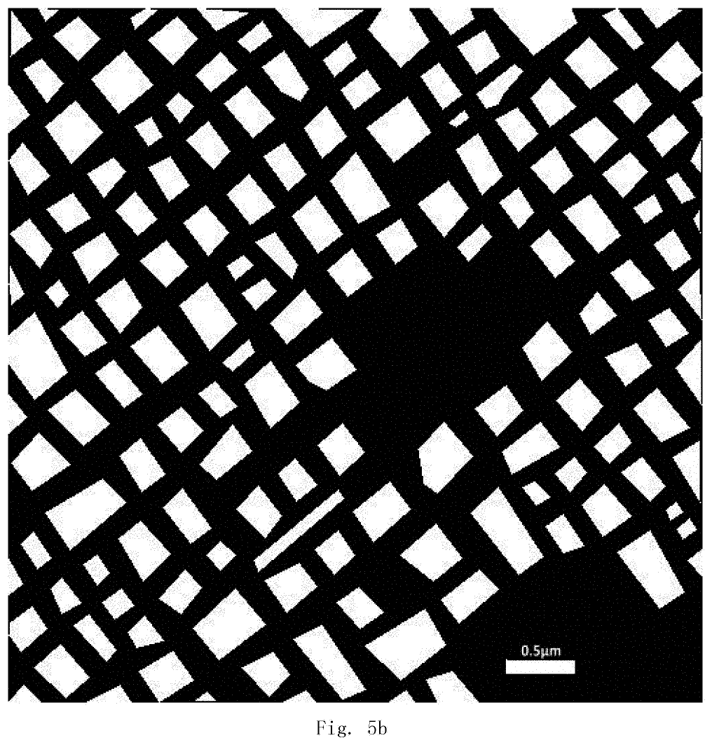

FIG. 6a shows images of features to be recognized and extracted;

FIG. 6b shows images of features extracted and recognized by the BD U-Net;

FIG. 6c shows partial regions in 6a;

FIG. 6d shows partial regions in 6b;

FIG. 7 shows a statistical result of single γ′ phase information;

FIG. 8a shows distribution of γ′ phases of small sizes in the full view field; and

FIG. 8b shows distribution of γ′ phases of large sizes in the full view field.

### DETAILED DESCRIPTION

The technical solution in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

The object of the present invention is to provide a full-view-field quantitative statistical distribution representation method for microstructures of γ′ phases in a metal material to realize automatic, high-speed and high-quality recognition and extraction of features of γ′ phases in the metal material and full-view-field in-situ quantitative statistical distribution representation of the features based on the depth learning theory, and overcome the defects of small view field, few features and insufficient representativeness of the traditional statistical method for γ′ phases.

To make the above-mentioned purpose, features and advantages of the present invention more clear and understandable, the present invention will be described below in detail in combination with the drawings and specific embodiments.

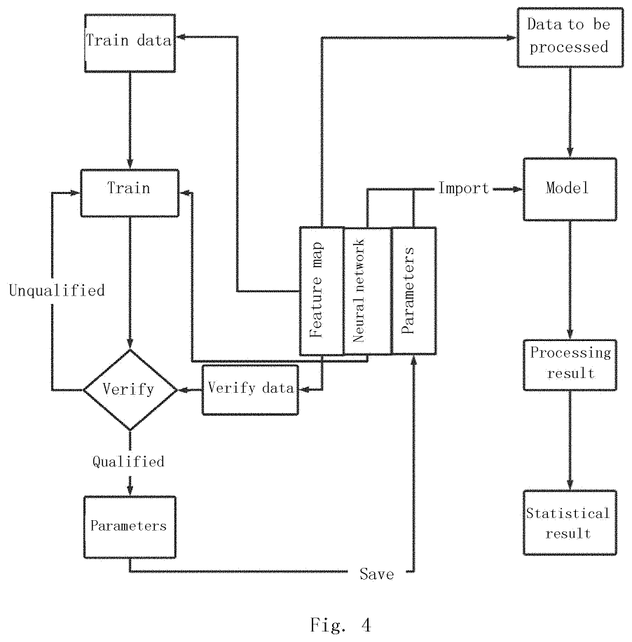

As shown in FIG. 4, the full-view-field quantitative statistical distribution representation method for microstructures of γ′ phases in a metal material provided by the present invention, comprising the following steps:

a) performing metallographic sample preparation on standard metal material samples with the same material as a metal material to be detected, to obtain a smooth metallographic mirror; performing chemical etching on the standard metal material samples, performing collection of γ′-phase feature maps on the standard metal material samples on which chemical etching is performed using a scanning electron microscope, and building a γ′-phase feature map data set; labeling the γ′ phases and cloud clutters as different features and the γ′ matrixes as background by Labelme, to obtain a labeled image containing the γ′ phases, cloud clutters and gamma matrixes, wherein the image only includes pixel gray values of three intensities, and different types of gray value intensities represent different features, and generating the labeled γ′-phase feature map data set into a feature sample set;

b) optimizing a deep learning-based image segmentation network U-Net, building a feature recognition and extraction network BDU-Net, performing data augmentation on the standard feature training samples, dividing the augmented data into a training set and a validation set, wherein the training set is used to train to obtain a feature recognition and extraction model, and the validation set is used to verify the reliability of the model; training with the training set, taking the MPA of the validation set as a judgment condition of training termination, saving parameters after the training is terminated, and saving the trained network as a final feature recognition and extraction model;

c) performing metallographic sample preparation, polishing and chemical etching on the metal material to be detected, and performing automatic collection of large-sized full-view-field γ′-phase feature maps on the surface of the processed metal material to be detected by a Navigator-OPA high-throughput scanning electron microscope, wherein the number of the automatically collected γ′-phase feature maps is more than 10000;

d) inputting the γ′ -phase feature maps obtained in the step c into the feature recognition and extraction model built in the step b, and thus obtaining binary images with γ′ phases labeled in situ; and

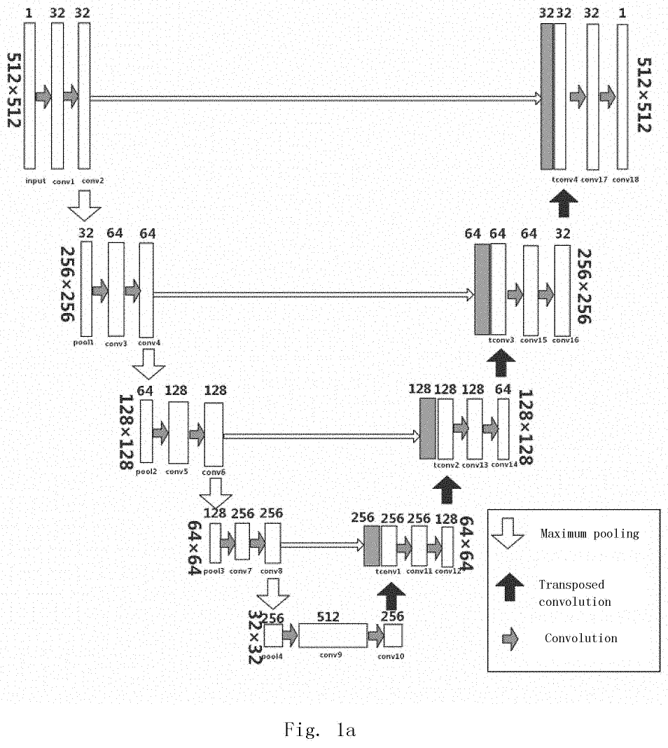

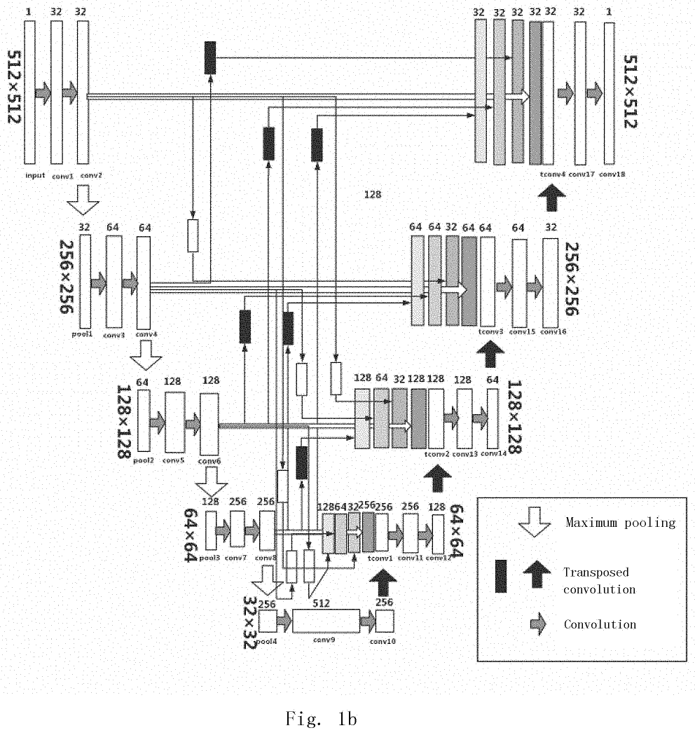

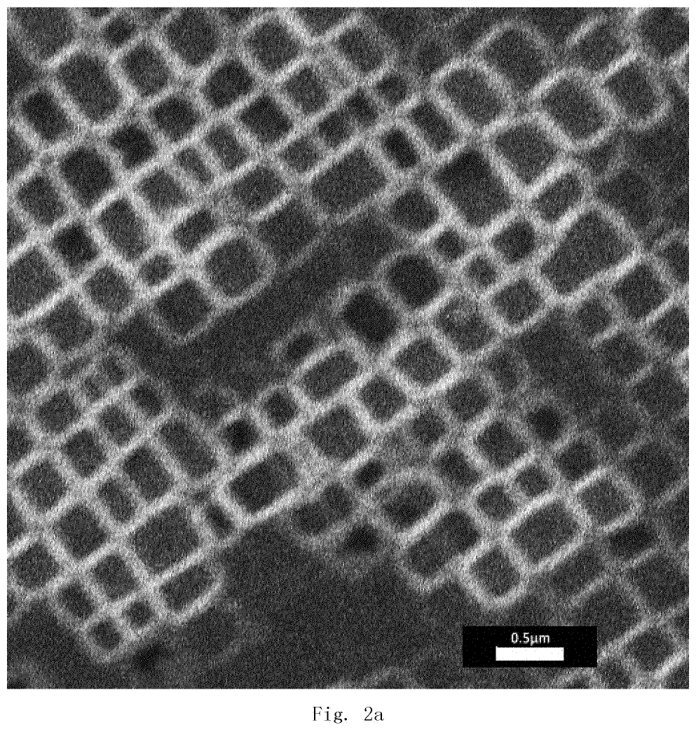

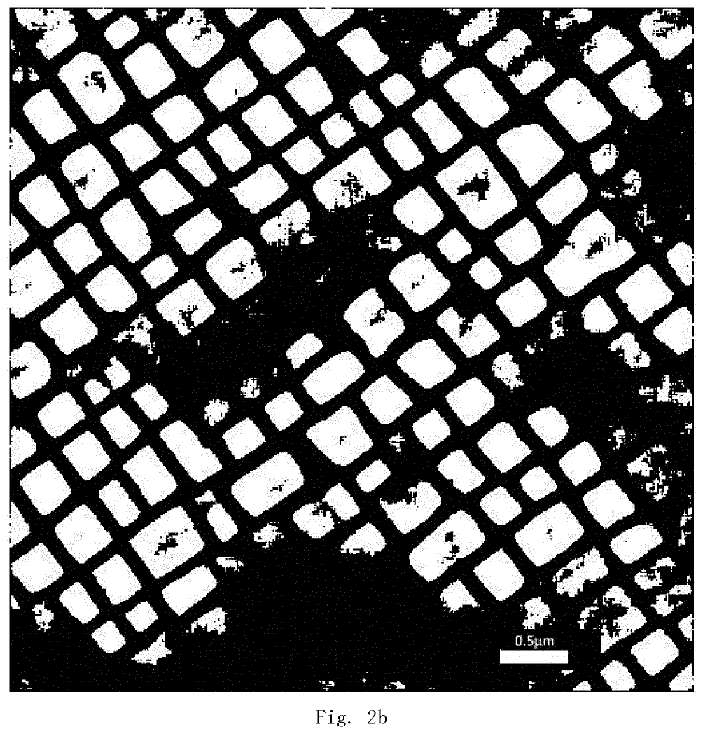

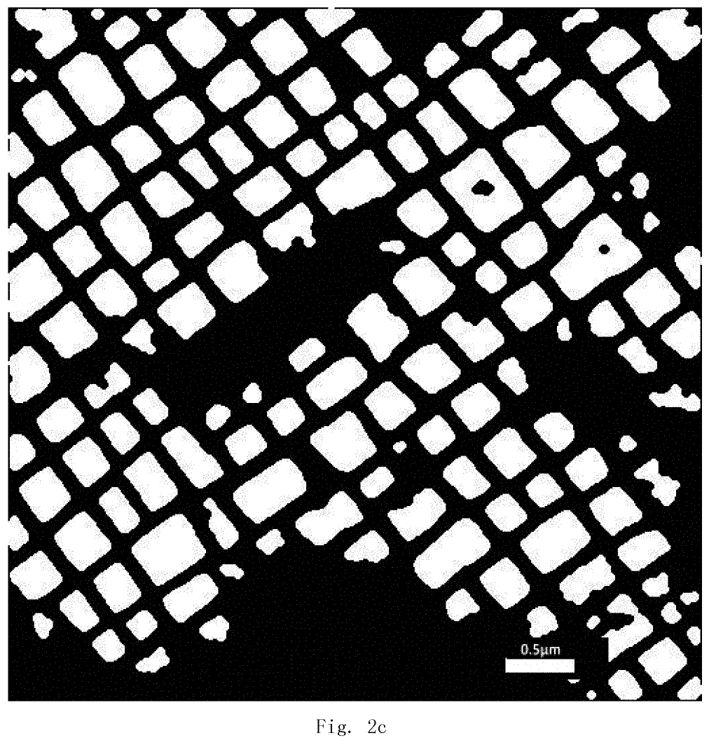

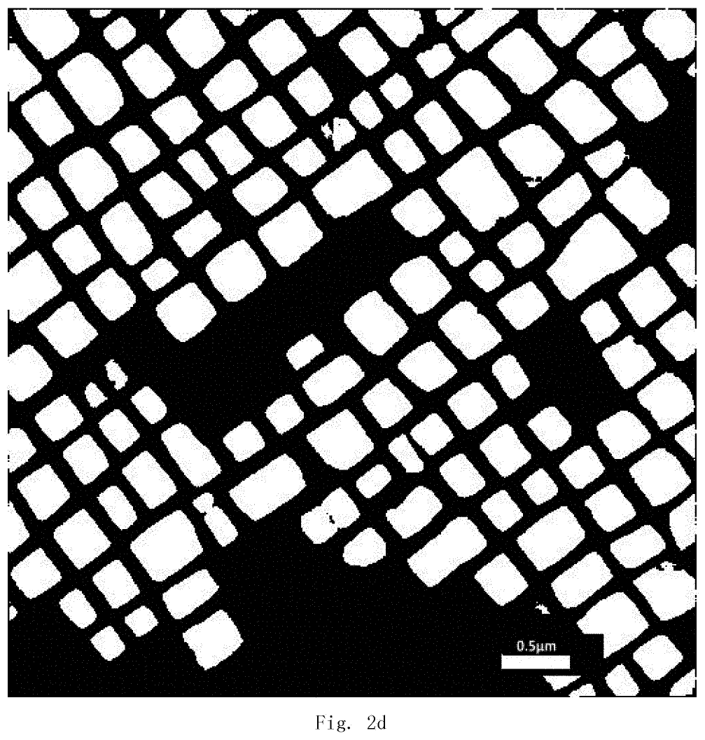

e) processing the binary images obtained in the step d by the connected component algorithm, acquiring the size, area and position information of each γ′ phase, mining the statistical results, selecting appropriate regions as calculation units, calculating the area fractions of γ′ phases of different sizes on each calculation unit, and studying the distribution of the γ′ phases of different sizes in the full view field.

Wherein in the step b, the feature recognition and extraction network is a Block-DenselJ-Net, the network including 9 blocks, the blocks being connected by a plurality of max-pooling layers and several transposed convolution layers, each block internally consisting of a plurality of convolution layers, ReLu activation functions and a Dropout layer, which respectively play the role of extracting deep-layer features from shallow-layer features, processing nonlinear problem and avoiding overfitting, wherein in the training process, the standard deviation, cross entropy and the like can be used as Loss functions. In the back propagation process, Adam optimization operators or Gradient-Descent operators may be selected as optimization functions. Compared with the U-Net, the BDU-Net has the advantages of integrating the concepts of the fully convolutional semantic, segmentation network U-Net and the DenseNet and focusing on strengthening the connection between blocks, for example, FIG. 1a is a diagram of the U-Net, FIG. 1b is a diagram of the BDU-Net, the improved network is obviously superior to the ordinary U-Net network in training speed and segmentation effect, as shown in the figure, FIG. 2a shows a test image, FIG. 2b shows a segmentation result obtained by the U-Net, FIG. 2c shows a result of post-processing the result obtained by the U-Net, and FIG. 2d shows a segmentation result obtained directly by the BDU-Net, i.e. the result shown in the figure, in the process of segmenting and extracting the γ′ phases, the BDU-Net is superior to the U-Net algorithm in effect.

In the step b, in order to avoid the overfilling caused by insufficient data in the training set, a data augmentation process is added before the start of training, through preprocessing methods such as translation, rollover, zooming-in/out, rotation and increase in noise, and random missing of sonic features of the original image, more real and comprehensive data information is simulated, the augmented data is trained, so the network can learn more comprehensive information, the trained model has stronger generalization ability, and thus more features obtained in different scenarios can be processed.

In the training process, the MPA (mean pixel accuracy) of the verification set is used as a judgment condition of training termination, the training termination threshold is set to 98% of the MPA, if the MPA of the verification set is greater than or equal to the termination threshold for three consecutive times, the training is terminated, and the trained network is saved as a final feature recognition and extraction model of this method.

Wherein in the step d, when the binary images of the γ′-phase feature maps are extracted using a view field with a pixel of 12288*42288, the time duration consumed in the extraction process is 12.5s.

Wherein in the step e, the size, area and position of 14400 γ′ phases are obtained respectively by means of the connected component algorithm, and are statistically analyzed, to obtain statistical results; the statistical results are mined, regions of 2.56 μm*2.56 μm are selected as calculation units, and the area fractions of the γ′ phases of different sizes on each calculation unit are calculated; in the step e, further comprising: visualizing the in-situ distribution of γ′ phases of different sizes in the full view field, and observing that the γ′ phases of small sizes are distributed in the dendrite trunk position and the γ′ phases of large sizes are distributed in the interdendritic position.

This embodiment describes a nickel-based single crystal superalloy for turbine blades of aeroengines. The directional solidification single crystal superalloy has excellent high-temperature strength, fatigue resistance, fracture toughness, and good oxidation and thermal corrosion resistance, thereby being a preferred material for turbine blades of aero-engines and gas turbines. A γ′ phase is the most important strengthening phase in the nickel-based single crystal superalloy, if the volume fraction of the γ′ phase is 65-70%, the durability of the alloy is greatly improved; moreover, the particle shape, size and solid solution element composition of γ′ phases have great influence on high-temperature creep performance; and on the other hand, the distribution of γ′ phases is closely related to the distribution of dendritic structures caused by the instability of the solid/liquid interface during the non-equilibrium solidification of the alloy. Therefore, the in-situ quantitative statistical distribution representation of γ′ phases in the single crystal superalloy and the non-unifoimity statistical distribution representation of the γ′ phases in the full view field are important basis for evaluating the process stability and reliability, and are of great significance for guiding the research of various properties of the single crystal superalloy.

When using the above-mentioned full-view-field quantitative statistical distribution representation method for microstructures of γ′ phases in a metal material, in the step a, standard bar sample of the nickel-based single crystal superalloy with matched composition (the composition includes: Cr: 5-6, Re: 2-3, Ta: 5-6, Al: 5-6, Co: 8.0-8.5, Mo: 0.4-0.6, W: 4-5, C: 0.01-0.02, B: 0.01-0.02, Hf: 0.1-0.2, Ni: balance) prepared by the directional solidification technology is coarsely ground, finely ground and finely polished with sandpaper to make a smooth metallographic mirror, and then is subjected to chemical etching in 1% HF, 33% HNO₃, 33% CH₃COOH, and 33% H₂O. The feature maps of the precipitated phases on the surface of the sample on which metallographic chemical etching is performed are randomly sampled and shot by a scanning electron microscope at, magnification of 10000 times, wherein the size of a single view field being 0.03 mm*0.03 mm, the pixel value of the single view field is 3072*3072, and the sampling position is random.

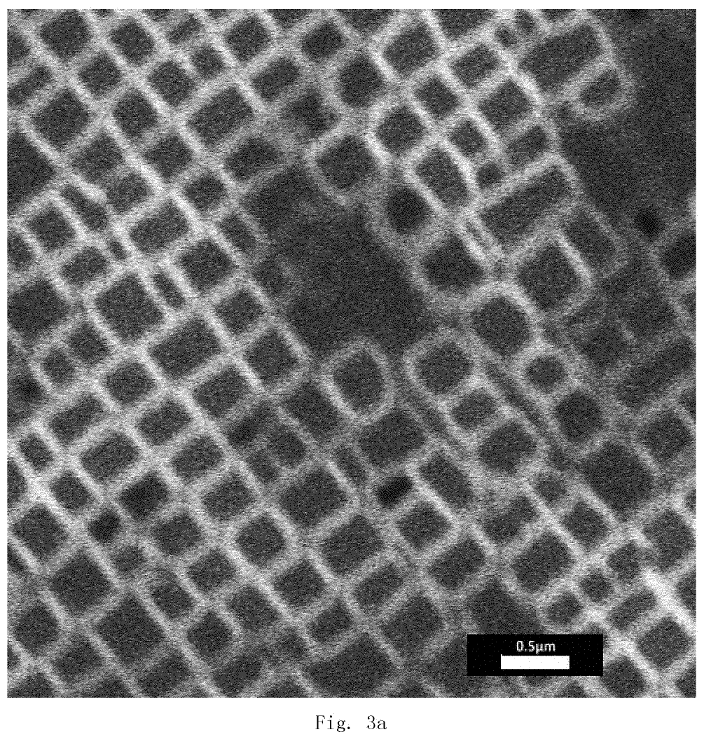

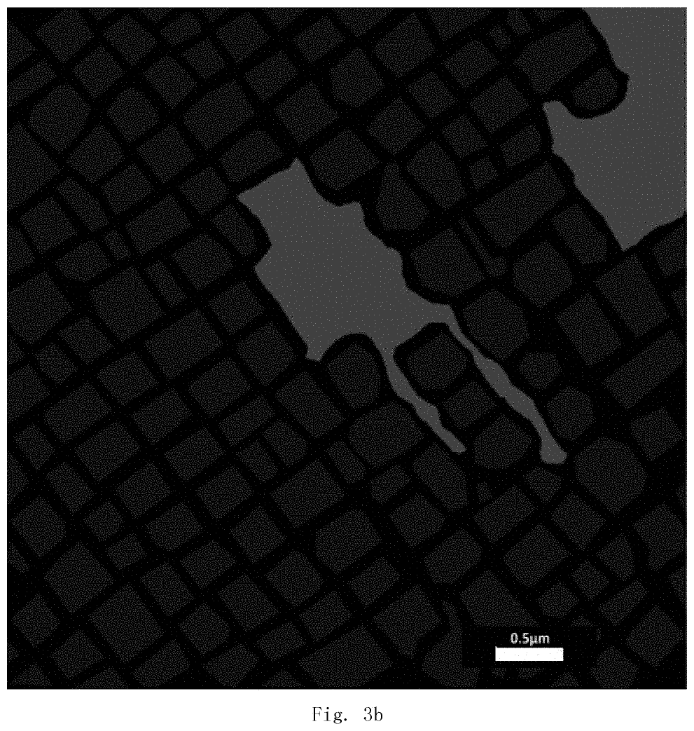

The collected feature maps are cropped, one view field is cropped into small view fields with a pixel of 512*512, and 300 small view fields are randomly selected from these small view fields and are manually labeled by Labelme to obtain a sample library for feature recognition and extraction. FIG. 3a shows a selected original image, and FIG. 3b shows a labeled image labeled by Labelme.

In the step b, as shown in FIG. 4, a flow chart of recognition, extraction and quantitative statistics of γ′-phase feature maps is made, and a DeepLeaming-based feature recognition and extraction network BDU-Net is built according to the flow chart, as shown in FIG. 1b. The network includes nine blocks respectively connected by ten max-pooling layers and ten transposed convolution layers, each block consisting of two convolution layers, two ReLu activation functions and one Dropout layer.

In step c, sample preparation and chemical etching are performed on the metal material whose γ′-phase features are to be extracted, and then automatic collection of full-view-field γ′-phase feature maps is performed on the surface of the metal material to be detected on which chemical etching is performed by a Navigator-®OPA high-throughput scanning electron microscope, wherein for a circular section with a diameter of 15 mm, the number of the automatically collected view fields is 120*120, i.e. the number of view fields in the X direction is 120, the number of view fields in the Y direction is 120, and feature maps of γ′ phases of 14400 view fields are obtained finally, each view field being an ultra-high resolution image with a pixel of 12288*12288.

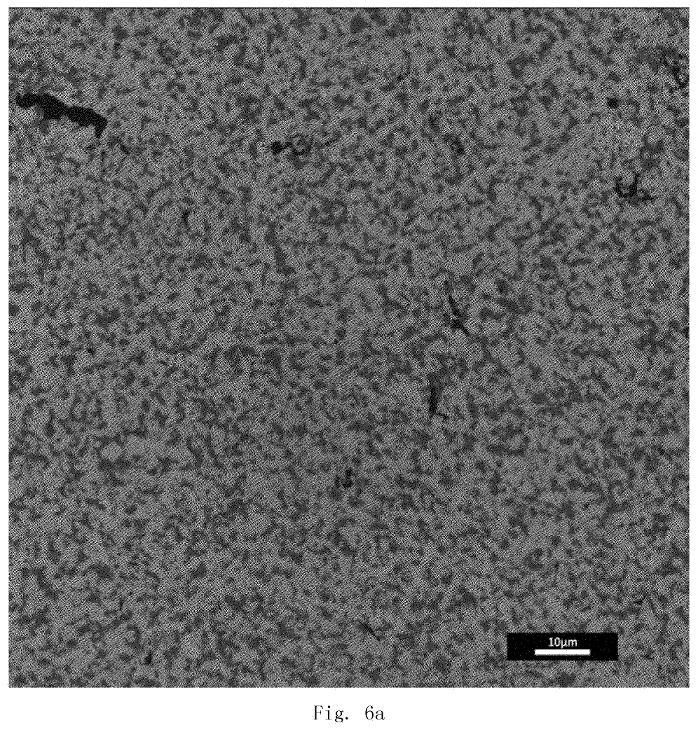

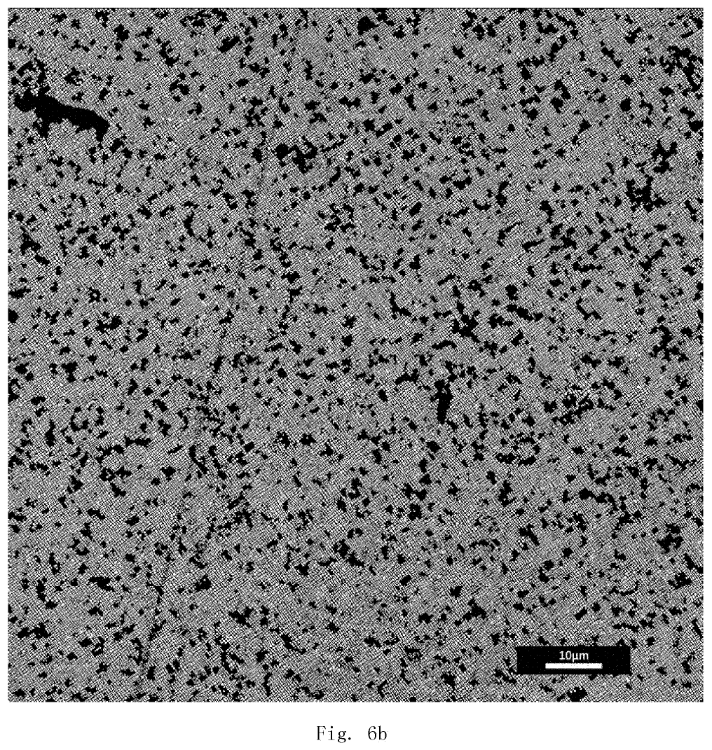

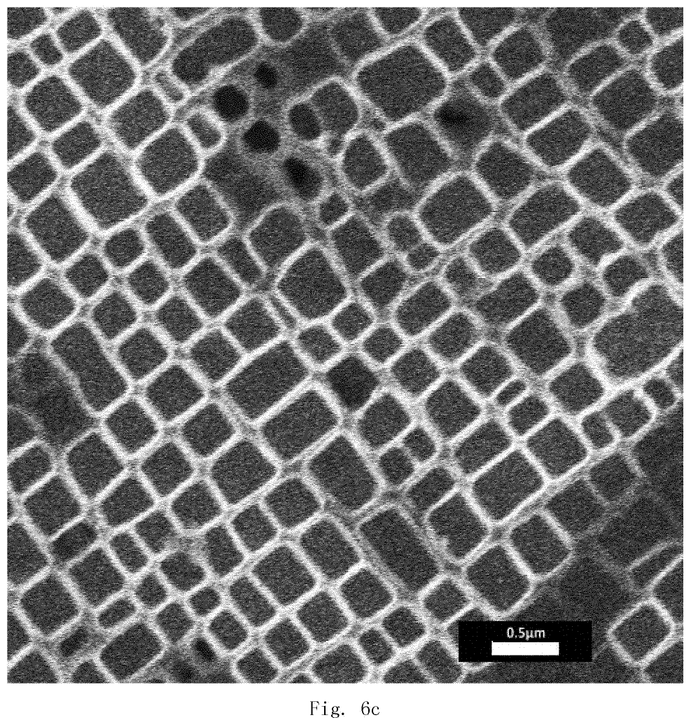

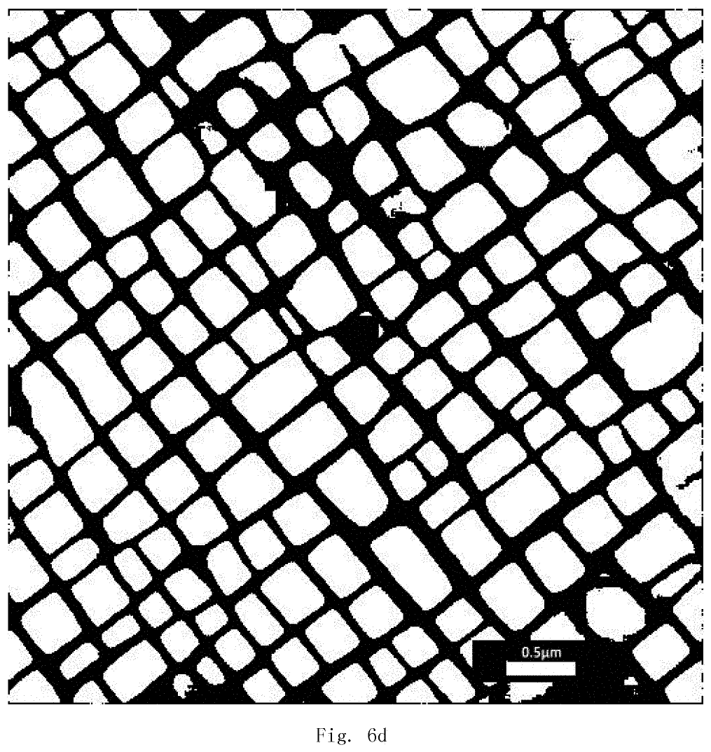

In step d, the images of all features to be recognized and extracted (as shown in FIG. 6a) are all input into the built feature recognition and extraction model for feature recognition and extraction, to obtain maps labeled with γ′-phase features as shown in FIG. 6b, wherein the time duration consumed for recognizing and extracting all the features in an, image as shown in FIG. 6a is 12.5s. FIG. 6c and FIG. 6d show partial, regions in FIG. 6a and FIG. 6b respectively.

In the step e, for the binary images with γ′-phase features labeled obtained in the step d, the size, area, and corresponding position in the full view field of each γ′ phase are acquired by means of the connected component algorithm. Further, according to the histogram of size distribution of all γ′ phases, an appropriate threshold is selected, the area fractions of γ′ phases in different sizes are calculated, and the distribution thereof is reflected in situ in the full view field.

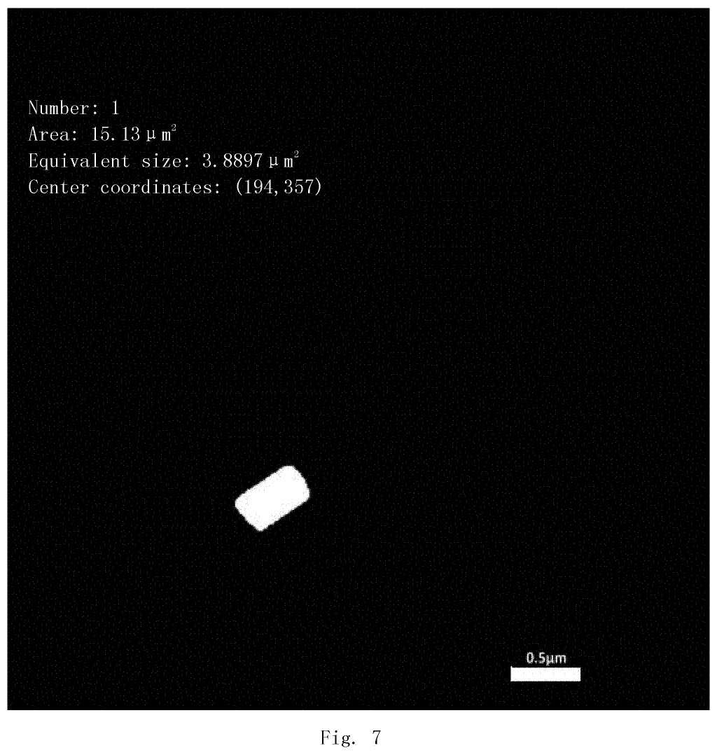

For each γ′ phase in the result, the area, equivalent size, position and other information are obtained by means of the connected component algorithm. FIG. 7 shows a schematic diagram showing statistics of single γ′ phase infothiation, and Table 1 shows summary of some statistical information of γ′ phases in the full total view field.

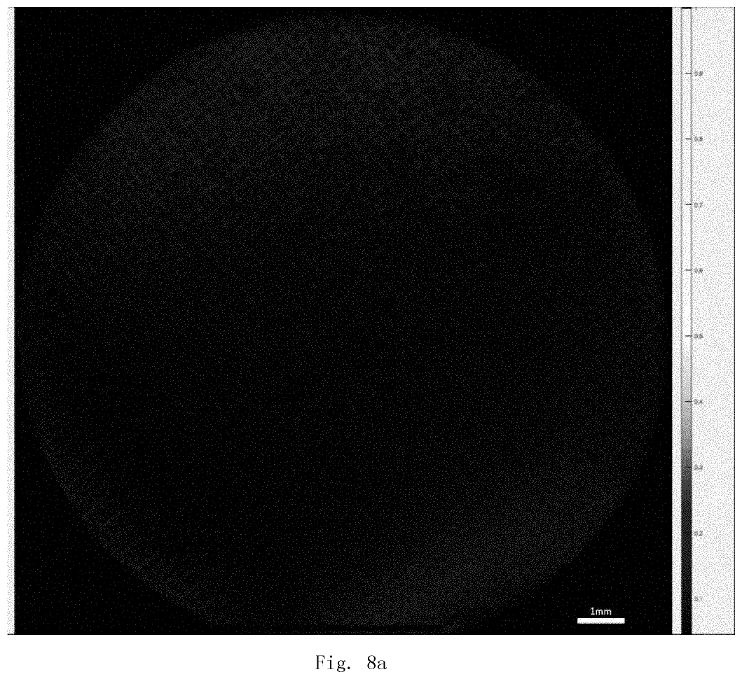

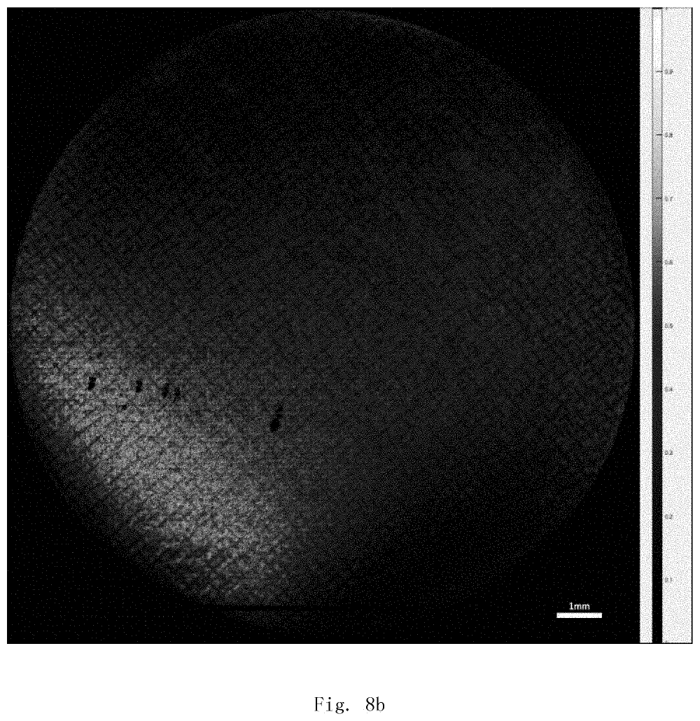

For all γ′ phases, the distribution of sizes thereof is counted. FIG. 7 is a histogram showing distribution of sizes. According to the histogram showing distribution of sizes, taking the peak as a threshold, the area fractions of γ′ phases in different sizes are respectively counted, so the distribution of γ′ phases of different sizes can be observed in the full view field. FIG. 8a shows the distribution of γ′ phases of small sizes in the full view field, and FIG. 8b shows the distribution of γ′ phases of large sizes in the full view field. It can be observed from the distribution of γ′ phases of different sizes in the full view field that the γ′ phases of small sizes are distributed in the dendrite trunk position and the γ′ phases of large sizes are distributed in the interdendritic position.

TABLE 1
Summary of statistical information of γ′ phases in full view field
Summary of statistical information of γ′ phases in full view field

| Area of full view | | Area fractionof γ′ |
|---|---|---|
| field (mm²) | Number of γ′ phases | phase (%) |
| 176.7146 | 904,574,619 | 62.282 |

By means of the full-view-field quantitative statistical distribution representation method for microstructures of γ′ phases in a metal material provided by the present invention, by building, a deep learning-based semantic segmentation neural network, after learning a few samples, a feature recognition and extraction model is obtained, so feature recognition and extraction work of a plurality of feature maps is completed quickly and efficiently at high quality, and in-situ quantitative statistical distribution representation is further realized in the full view field. The feature recognition and extraction work in the present invention is realized by means of the BDU-Net (Block-DenseU-Net) semantic segmentation algorithm, the algorithm having the characteristics of good effect, fast speed, and strong generalization ability in the process of feature recognition and extraction, solving the problems of excessive dependence on manual labor and low efficiency in the process of recognition and extraction of the microstructures of traditional metal materials. By means of the full-view-field in-situ quantitative statistical method of the present invention, the detailed information of each microstructure is quantitatively counted on an in-situ basis and the phenomenon of insufficient representativeness due to the fact that statistical analysis is only performed on partial information in traditional method is avoided. The method has the characteristics of automation, high quality, high speed and comprehensiveness, greatly improves the representation efficiency of microstructures, and meets the requirements of material genetic engineering for high-throughput representation of material microstructures.

Specific individual cases are applied herein for elaborating the principle and embodiments of the present invention. The illustration of the above embodiments is merely used for helping to understand the present invention and the core thought thereof. Meanwhile, for those ordinary skilled in the art, specific embodiments and the application scope may be changed in accordance with the thought of the present invention. In conclusion, the contents of the description shall not be interpreted as a limitation to the present invention.

1. A full-view-field quantitative statistical distribution representation method for microstructures of γ′ phases in a metal material, comprising the following steps:
a) performing metallographic sample preparation, polishing and chemical etching on standard metal material samples with the same material as a metal material to be detected, randomly sampling and shooting the processed standard metal material samples by a scanning electron microscope at high magnification, and building a γ′-phase feature map data set; labeling γ′ phases, cloud clutters and γ matrixes by Labelme, and then making standard feature training samples;
b) optimizing a deep learning-based image segmentation network U-Net, building a feature recognition and extraction network BDU-Net, performing data augmentation on the standard feature training samples, dividing the augmented data into a training set and a validation set, training with the training set, taking the MPA of the validation set as a judgment condition of training termination, saving parameters after the training is terminated, and saving the trained network as a final feature recognition and extraction model;
c) performing metallographic sample preparation, polishing and chemical etching on the metal material to be detected, and performing automatic collection of large-sized full-view-field γ′-phase feature maps on the surface of the processed metal material to be detected by a Navigator-OPA high-throughput scanning electron microscope;
d) inputting the γ′-phase feature maps obtained in the step c into the feature recognition and extraction model built in the step b, and thus obtaining binary images with γ′ phases labeled in situ; and
e) processing the binary images obtained in the step d by means of the connected component algorithm, acquiring, the size, area and position information of each γ′ phase, mining the statistical results, selecting appropriate regions as calculation units, calculating the area fractions of γ′ phases of different sizes on each calculation unit, and studying the distribution of the γ′ phases of different sizes in the full view field.

2. The full-view-field quantitative statistical distribution representation method for microstructures of γ′ phases in a metal material according to claim 1, wherein in the step c, the number of the automatically collected γ′-phase feature maps is more than 10000.

3. The full-view-field quantitative statistical distribution representation method for microstructures of γ′ phases in a metal material according to claim 1, wherein in the step b, the feature recognition and extraction network is a new feature recognition network BDU-Net proposed by adding a connection between blocks on the basis of the U-Net, the BD-U-Net including nine blocks respectively connected by ten maximum pooling layers and ten transposed convolution layers, each block internally consisting of two convolution layers, two ReLu activation functions and one Dropout layer.

4. The full-view-field quantitative statistical distribution representation method for microstructures of γ′ phases in a metal material according to claim 1, wherein in the step b, further comprising: preprocessing images containing γ′ phases in the standard feature map data set, specifically including translation, rollover, zooming-in/out, rotation and increase in noise.

5. The full-view-field quantitative statistical distribution representation method for microstructures of γ′ phases in a metal material according to claim 1, wherein in the step d, when the binary images of the -y -phase feature maps are extracted using a view field with a pixel of 12288*12288, the time duration consumed in the extraction process is 12.5s.

6. The full-view-field quantitative statistical distribution representation method for microstructures of γ′ phases in a metal material according to claim 1, wherein in the step e, the size, area and position of 14400 γ′ phases are obtained respectively by means of the connected component algorithm, and are statistically analyzed, to obtain statistical results.

7. The full-view-field quantitative statistical distribution representation method for microstructures of γ′ phases in a metal material according to claim 1, wherein in the step e, the statistical results are mined, regions of 2.56 μm * 2.56 μm are selected as, calculation units, and the area fractions of the γ′ phases of different sizes on each calculation unit are calculated.

8. The full-view-field quantitative statistical distribution representation method for microstructures of γ′ phases in a metal material according to claim 1, wherein in the step e, further comprising: visualizing the in-situ distribution of γ′ phases of different sizes in the full view field, and observing that the γ′ phases of small sizes are distributed in the dendrite trunk position and the γ′ phases of large sizes are distributed in the interdendritic position.
